(19) United States
(12) Patent Application Publication
(10) Pub. No.: US 2006/0240409 A1
(43) Pub. Date: Oct. 26, 2006

(54) Method for extraction and identification of nucleic acids

(76) Inventors: Alfred M. Prince, Pound Ridge, NY (US); Wolfram H.E. Pfahler, Pelting (DE); Don-Hun Lee, River Egde, NJ (US); Linda Andrus, New York, NY (US)

Correspondence Address: HOFFMANN & BARON, LLP, 6900 JERICHO TURNPIKE, SYOSSET, NY, 11791, US

(21) Appl. No.: 11/336,620

(22) Filed: Jan. 20, 2006

Related U.S. Application Data
(60) Provisional application No. 60/645,905, filed on Jan. 21, 2005.

(51) Int. Cl. C12Q 1/70; C12Q 1/68; C07H 21/02; C07H 21/04

(52) U.S. Cl. 435/5; 435/6; 536/25.4

(57) ABSTRACT

The invention provides a method for extracting nucleic acids from a sample. The sample contains cells, viruses, or both cells and viruses. The method include adding a lysing solution containing a detergent to the sample to lyse the cells or viruses to form a lysate, adding alcohol to the lysate to aggregate or precipitate the nucleic acids, and purifying the nucleic acids from the lysate-alcohol mixture by filtering the mixture through a glass-fiber filter.

This application asserts priority to U.S. Provisional Application Ser. No. 60/645,905 filed on Jan. 21, 2005, the specification of which is incorporated by reference in its entirety.

### BACKGROUND OF THE INVENTION

The public health sector increasingly demands highly sensitive assays for viruses, bacteria, fungi, parasites, or cellular genes. High throughput sample processing for screening (e.g., blood supply, arbo-viruses in mosquitoes), surveillance (e.g., West Nile Virus in bird populations), analysis of water, diagnosis of infections, gene based diagnosis (e.g., for hemophilia, predisposition for breast cancer, cancerous cells), etc. would be beneficial.

Contamination of the blood supply with pathogenic viruses, such as human immunodeficiency virus (HIV), hepatitis A, B or C virus, parvovirus, cytomegalovirus and Epstein Barr virus, and bacterial infections, such as Lyme disease, has become an increasingly serious problem. The prevailing opinion of the U.S. Food and Drug Administration and elsewhere is that all blood should be screened using polymerase chain reaction (PCR) analysis in addition to, or eventually to replace, serological tests. It is thought that screening blood for infectious viruses will prevent at least one hundred transfusion-associated cases of hepatitis B virus (HBV), hepatitis C virus (HCV), and HIV per year.

Serological tests were until recently the method of choice for screening blood. These tests detect the presence in the blood of antibodies raised against viral agents, viral antigens, bacterial agents, bacterial antigens, etc. Serological screening tests have the drawback of not being able to detect an infection if an antibody response has not been mounted.

For example, serological tests often fail to detect infected individuals during the early stages of infection. In addition, individuals who exhibit low immune responses generally harbor a small amount of virus. Typically, the small amount of viruses do not stimulate the production of antibodies. An example of such viruses is HIV. Because of these and other practical limitations to serological testing, there is a need for methods that will detect infections regardless of an individual's stage of infection.

Isolating nucleic acids present in the blood plasma followed by PCR amplification enables the detection of pathogenic agents in the absence of antibodies. The detection of pathogenic agents is crucial to insure that the blood supply is free from transmissible pathogens.

The screening of blood and related biological materials in a medical setting is usually performed on a massive scale. Blood centers commonly test as much as one thousand or more units of blood each day. The preparation of isolated nucleic acids from a thousand samples of blood per day using the presently available techniques require considerable amounts of time, labor and reagents. Thus, large-scale nucleic acid testing of individual samples is generally not performed because of technical limitations.

Currently, nucleic acid testing in screening and surveillance applications is used, if at all, in pools of samples. Pooled samples, unfortunately, reduce the sensitivity of the tests. If a pooled sample tests positive, the final diagnosis is delayed.

Extracting DNA or RNA for testing has generally involved the use of two different extraction methods. One method allows only for the extraction of DNA; the other method allows only for the extraction of RNA. Use of the DNA extraction method results in poor yield of RNA, and vice versa. Thus, until recently, blood screening required one procedure to isolate DNA, and a different procedure to isolate RNA.

Accordingly, there is a need for a simple, efficient and reliable method which allows highly sensitive extraction and purification of both DNA and RNA. Such a method is especially useful for screening the blood supply. The method is also beneficial for numerous other applications, such as gene based diagnosis, surveillance of infectious disease (e.g., West Nile virus in bird populations, malaria in mosquitoe populations), analysis of water, etc.

### SUMMARY OF THE INVENTION

The above need has been met by the present invention which provides a method for extracting nucleic acids from a sample. The method comprises obtaining a sample containing cells, viruses, or both cells and viruses; adding a lysing solution comprising a detergent to the sample, thereby lysing the cells or viruses and forming a lysate; adding an amount of alcohol to the lysate sufficient to aggregate or precipitate nucleic acids; and purifying the nucleic acids from the lysate-alcohol mixture by filtering the mixture through a glass-fiber-filter.

In another embodiment, the invention provides a method for identifying a pathogen in a sample. The method comprises obtaining a sample containing cells, viruses, or both cells and viruses; adding a lysing solution comprising a detergent to the sample, thereby lysing the cells or viruses and forming a lysate; adding an amount of alcohol to the lysate sufficient to aggregate or precipitate nucleic acids; purifying the nucleic acids from the lysate-alcohol mixture by filtering the mixture through a glass-fiber-filter; and assaying the nucleic acids to identify the pathogen.

In yet another embodiment, the invention provides a method for identifying biological contaminants in a water sample. The method comprises obtaining a water sample containing cells, viruses, or both cells and viruses; adding a lysing solution comprising a detergent to the sample, thereby lysing the cells or viruses and forming a lysate; adding an amount of alcohol to the lysate sufficient to aggregate or precipitate nucleic acids; purifying the nucleic acids from the lysate-alcohol mixture by filtering the mixture through a glass-fiber-filter; and assaying the nucleic acids to identify the contaminants.

In a further embodiment, the invention provides a method for identifying a genetic disorder in a mammal. The method comprises obtaining a biological sample containing cells; adding a lysing solution comprising a detergent to the sample, thereby lysing the cells or viruses and forming a lysate; adding an amount of alcohol to the lysate sufficient to aggregate or precipitate nucleic acids; purifying the nucleic acids from the lysate-alcohol mixture by filtering the mixture through a glass-fiber-filter; and assaying the nucleic acids to identify the genetic disorder.

In another embodiment, the invention provides a kit for extracting nucleic acids from a sample. The kit comprises a lysing solution comprising a detergent and glass-fiber filters.

### BRIEF DESCRIPTION OF THE FIGURES

FIG. 1: Effect of PEG on virus detection. Normal human plasma was spiked with 104.5 WNV genome equivalents (GE) per milliliter. PEG 8000 was added at various concentrations. 2.0 ml of PEG-plasma were mixed and centrifuged. 200 μl of each, precipitate and supernatant, were submitted to extraction and quantitative RT-PCR.

FIG. 2: Effect of PEG on virus detection. 200 μl of non-concentrated and PEG concentrated plasma were extracted and subjected to PCR. Compared to 0% PEG, approximately 10 times more RNA could be detected at 3% PEG.

FIG. 3. WNV Stability in Plasma at 4° C. Endpoint RT-PCR was performed on RNA extracted from WNV samples, which were stored at 4° C. for 0, 7 or 14 days. Results are expressed as mean relative fluorescence units (RFU)+/−standard deviation of 4 replicate samples.

### DETAILED DESCRIPTION OF THE INVENTION

The invention is based on the surprising discovery by the inventors of a method for rapid and efficient extraction of both DNA and RNA from samples containing both DNA and RNA simultaneously, i.e. using one procedure. It has unexpectedly been found that both DNA and RNA can be separated, i.e. purified, from samples by lycing the sample with detergent, aggregating or precipitating any nucleic acids present by adding an alcohol, and separating the nucleic acids from the lysate by filtering the mixture through a glass fiber filter.

The extraction and purification procedure is suitable for automation. The extracted nucleic acids are compatible with nucleic acid amplification techniques, such as PCR (polymerase chain reaction) or RT-PCR (reverse transcription-polymerase chain reaction).

Extracting Nucleic Acids

In one embodiment, the invention provides a method for extracting nucleic acids from a sample. The nucleic acids include deoxyribonucleic acids (DNA) or ribonucleic acids (RNA).

The first step in the method for extracting nucleic acids from a sample is to obtain a sample containing cells or viruses. Any sample containing cells or viruses can be employed in accordance with the methods of the present invention. Examples of samples which contain cells or viruses include, but are not limited to, biological samples and aqueous non-biological samples.

Any biological sample containing cells or viruses is suitable for use in the method of the present invention. A biological sample as used herein includes, for example, body fluids, tissues and cells. Some specific examples of biological samples include, but are not limited to, blood, blood plasma, urine, saliva, vaginal fluid, cerebral spinal fluid, blood serum, epithelial cells, immune cells, buccal scrapings, cervical tissue scrapings, etc.

The biological sample can be obtained by any method known to those in the art. Suitable methods include, for example, venous puncture of a vein to obtain a blood sample and cheek cell scraping to obtain a buccal sample.

The sample can contain cells. The cells can be any cell known to those in the art. The term “cells” as used herein includes individual cells and cells that are part of tissue. The cells may be present in body fluids. Individual cells include, among others, the cells mentioned above (e.g., epithelial cell, immune cells, etc.).

The term “cells” also include microorganisms, in whole or in part. The microorganism is typically pathogenic (i.e., causes disease), but may be non-pathogenic. Examples of microorganisms include, bacteria, parasites, fungi, algae, and the like.

The bacteria can be any bacteria known to those skilled in the art. Some examples of bacteria include, Borrelia species, Leptospir species, Mycobacteria species, etc.

Parasites are organisms that grow, feed, and are sheltered on or in a different organism while typically having an adverse effect on the survival of its host. The nucleic acids from any parasite known to those in the art can be extracted in accordance with the methods of the present invention. Some examples of parasites include, Schistosoma species, Leishmania, species, Trichomonas species, Plasmodium species (e.g., malaria), Toxoplasma species, Cryptosporidium species, and Entameoba species, etc.

Fungi are eukaryotic organisms which lack chlorophyll and vascular tissue, and generally range in form from a single cell to a body mass of branched filamentous hyphae. Any fungi known to those in the art can be used in accordance with the methods of the present invention. Some examples of fungi include molds and yeast (e.g., Candida species, Saccharomyces species, etc.).

Algae are generally aquatic, eukaryotic, photosynthetic organisms. The algae can be any algae known to those skilled in the art. Examples of algae include, but are not limited to cyanobacteria.

The sample can, in addition, contain viruses. The nucleic acids from any virus known to those in the art can be extracted in accordance with the methods of the present invention. Such viruses include DNA viruses and RNA viruses.

Examples of DNA viruses include poxvirus, herpesvirus, adenovirus, papovavirus, hepadnavirus (e.g., hepatitis B virus) and parvovirus (e.g., parvovirus B19 virus). Examples of RNA viruses include picornavirus (e.g., hepatitis A virus), calcivirus, togavirus, flavivirus (e.g., hepatitis C virus and West Nile virus), coronavirus, reovirus, rhabdovirus, filovirus, paramyxovirus, orthomyxovirus, bunyavirus, arenavirus, and retroviruses (e.g., human immunodeficiency virus).

The sample can be obtained from any organism, or can be an entire organism if the organism is of a suitably small size. Examples of suitable organisms include microorganisms, and tissue body fluids from mammals, birds, aquatic animals (e.g., fish), or arthopods (e.g., ticks, mosquitoes, etc.), and fungi. Micororganisms include those described above.

The mammal can be any mammal known to those skilled in the art. Mammals include, for example, humans, baboons, and other primates, as well as pet animals such as dogs and cats, laboratory animals such as rats and mice, and farm animals such as horses, sheep, and cows.

In one embodiment, the sample is an aqueous non-biological sample suspected of being contaminated with cells and/or viruses. The sample can be obtained from, for example, drinking water and bodies of water (e.g., lakes, streams, rivers, oceans, etc.) Extracting nucleic acids from cells or viruses in a water sample is useful for testing, for example, drinking water for contaminants, as discussed below.

Those skilled in the art will appreciate that extraction of nucleic acids from the various types of samples may require different types of sample preparation so as to prepare the sample for use in the method of the present invention. Some examples of suitable preparation techniques include the addition of different types of buffer systems, solutions, etc.

Anther suitable preparation technique includes the elimination of cells from the sample. For example, in order to prepare a blood serum sample, cells are usually, but not necessarily, eliminated from the sample, such as from a whole blood sample. The cells can be eliminated from the sample by any method known to those skilled in the art. For example, centrifugation or filtration can be used to prepare a cell-free sample. For instance, blood serum is generally obtained from clotted blood by centrifugation to remove cellular components. Plasma is usually obtained in a similar manner as blood serum except that an anticoagulant is added to the blood.

In another embodiment, the method optionally further comprises the step of concentrating the cells or viruses in the sample. Any concentration method known to those in the art can be employed. Suitable concentration methods include, but are not limited to the use of, polyethylene glycol.

An appropriate concentration for use in the method typically will partly depend on the nature of the sample. The concentration method may, for example, depend on whether the sample contains cells, viruses, or both; on the type of cells; etc.

In a first embodiment, samples, including samples containing viruses, are concentrated with polyethylene glycol. Any polymer of polyethylene glycol can be useful in the method of the present invention. For example, the polyethylene glycol may have a minimum molecular weight of about 120, preferably about 5,000 and more preferably about 7,000. The maximum molecular weight of the polyethylene glycol may be about 10,000, preferably about 9,500, and more preferably about 9,000. Any of the above minima and maxima can be combined to provide a suitable range for the polyethylene glycol. Preferably, the polyethylene glycol has a molecule weight of about 8,000.

In a second embodiment, ammonium sulfate is used to concentrate cells or viruses. Suitable concentrations of ammonium sulfate for use in the methods of the present invention may be, for instance, between about 5% and about 50% v/v.

In a third embodiment, centrifugation and/or ultracentrifugation is used to concentrate cells or viruses in a sample. Appropriate centrifugation conditions (e.g., time, speed, temperature, etc.) can be determined by those skilled in the art. Typically, centrifugation is for concentrating cells, while ultracentrifugation is usually employed for concentrating viruses.

In the second step of the method, a lysate is formed by adding a lysing solution comprising a detergent to the sample. The lysing solution is added in an amount sufficient to lyse the cells or viruses. “Lyse” as used herein generally refers to the physico-chemical disruption of the structural components (e.g., viral envelope and capsid, cell membrane, coagulated proteins, etc.) of the cells or virus.

The lysing solution useful in the method of the present invention comprises a detergent capable of solubilizing lipids. Detergents include, but are not limited to, sodium dodecyl sulfate (i.e., sodium lauryl sulfate), tri-N-butylphosphate, Brij-35, octyl β-glucoside, octyl β-thioglucopyranoside, and the like.

The lysing solution is contacted with the sample by any method known to those in the art. Typically, the lysing solution is incubated with the sample for a sufficient time and temperature to disrupt the cells and/or viruses. Generally, the lysing solution is pipetted into the sample. Suitable incubation conditions (e.g., time, temperature, etc.) can be readily determined by those skilled in the art. Generally, the sample is incubated with the lysing solution for at least one or more minutes usually at temperatures between 4° C. and 90° C., with or without agitation. Examples of agitation include, but are not limited to, shaking, stirring, vibrating, vortexing, or any other type of mechanical blending.

The concentration of detergents in the lysing solution will depend on various factors, such as, for example, strength of the detergent, incubation conditions, etc. For example, detergent concentrations generally range from about 0.1% to about 10% v/v.

In one embodiment, the lysing solution further comprises a proteinase. Typically, proteinases are useful for digesting proteins. Proteinases are particularly useful in the method of the present invention for samples containing cells or viruses in which the nucleic acids are associated with proteins. An example of such a virus is the hepatitis B.

Any proteinase known to those in the art can be employed. Examples of suitable proteinases include proteinase K, pepsin, trypsin, chymotrypsin, and the like. The minimum amount of proteinases in the lysing solution is generally about 0.1 mg/ml, preferably about 0.4 mg/ml, and more preferably about 0.7 mg/ml. The maximum amount of proteinases in the lysing solution is typically about 10 mg/ml, preferably about 7 mg/ml and more preferably about 5 mg/ml. Any of the above minima and maxima can be combined to provide a range for the proteinase. Usually, the lysing solution contains about 1 mg/ml of proteinase.

After a lysate is formed, the next step in the method for extracting nucleic acids is to add alcohol to the lysate, especially a water soluble alcohol. Any alcohol known to those skilled in the art can be used in accordance with the methods of the present invention. Examples of alcohols include, but are not limited to, ethanol, isopropanol, and the like. Another example of a useful alcohol is methanol. The addition of alcohol to the lysate generally results in aggregation or precipitation of the nucleic acids from solution.

The concentration and amount of alcohol added to the lysate, may be any concentration and amount sufficient to aggregate or precipitate the nucleic acids. Such concentrations and amounts can be readily determined by those skilled in the art.

The actual amount of alcohol will vary according to various factors well known in the art, such as the particular alcohol utilized, the concentration of alcohol to be added, the volume of the lysate solution, and the type and preparation of the sample subjected to lysis. The alcohol added to the lysate can be 100% alcohol, or a solution of alcohol and water, such as a 50%, 70% or 95% alcohol solution. For example, for 100% alcohol, the amount of alcohol added is about one-tenth to about two times the volume of the lysate solution, and optimally about one-half the volume of the lysate solution.

The final step in the method for extracting nucleic acids comprises purifying the nucleic acids from the lysate-alcohol mixture. The nucleic acids are purified by separating the nucleic acids from the non-nucleic acid portion of the lysate-alcohol mixture by filtering the mixture through a glass-fiber filter. These filters are micorofiber filters manufactured from borosilicate glass. The glass-fiber filters allow for high flow rates and high binding capacity. Suitable glass-fiber filters include, for example, type GF/F commercially available from Whatman, Clifton, N.J.

The minimum pore size of the glass-fiber filter is about 0.4 μm and preferably about 0.6 μm. The maximum pore size is about 1.2 μm, preferably about 1.0 μm, and more preferably about 0.8 μm. Any of the above minima and maxima can be combined to provide a suitable range for the filter's pore size. Preferably, the pore size is about 0.7 μm.

The lysate-alcohol mixture is permitted to pass through the filter. The flow of the lysate-alcohol mixture may be promoted by the application of a force. For example, apparatuses that provide a negative pressure beneath the filter, or a positive pressure above the filter, can be used to provide the necessary force to assist the alcohol solution to pass through the filter.

The filtering step can, for example, utilize a multiple well filtration plate fitted into a vacuum manifold. The filtration plates have as their filter components the glass-fiber filters of the method of the invention. Filtration plates suitable for use are commercially available. For example, 96-well glass-fiber filter plates type GF/F are commercially available from Whatman, Clifton, N.J. Plates containing more or fewer than 96 wells are also suitable, and can be prepared and implemented depending upon the needs of the user.

Vacuum manifolds, designed to accommodate multiple-well filtration plates, are commercially available and are used routinely to process multiple samples. For example, a vacuum manifold furnished by Millipore can be used. The multiple-well filtration plate is situated such that the plate sits on a manifold plate support with a sealing gasket around its edge.

The use of multi-well plates, such as 96- or 386-well plates, allows the processing of many samples at the same time. When such plates are fitted to a vacuum manifold, samples can be passed through all the wells simultaneously. Thus, multiple samples may be processed at the same time. Accordingly, the method of the invention is adaptable to automation using laboratory robotics.

For example, samples can be processed using a robotic liquid handling system in conjunction with a vacuum unit to draw the samples through each of the wells simultaneously. The capacity for automating the extraction of nucleic acid is a valuable advantage in, for example, screening where many samples need to be processed rapidly, such as blood or genetic screening.

Other forces which can be applied to assist in the flow or passage of the lysate-alcohol mixture through the glass-fiber filter include the use of positive pressure from the top of the filter plate, centrifugation or gravity. Multi-well plates can be used with specially designed centrifuge systems using plate rotors to process numerous samples simultaneously.

After purifying the nucleic acids from the lysate-alcohol mixture by filtering the mixture through a glass-fiber filter, the nucleic acids bound to the glass-fiber filter are optionally washed with a washing buffer. A pressurizing apparatus may be employed to assist the flow or passage of the washing buffer through the glass-fiber filter. Alternatively, vacuum, centrifugation or gravity can be used to assist the passage of the washing buffer through the filter.

The washing buffer can be any nucleic acid washing buffer known to those skilled in the art. A preferred washing buffer is a solution comprising about 10% to about 100% ethanol, optimally about 50% to about 70% ethanol. The washing buffer further comprises about 10 to about 1000 mM NaCl, 10 mM Tris-HCL and 2 mM EDTA, and optimally 100 mM EDTA. Other salts, buffers, chelating agents and alcohols known to those in the art are suitable. The washing buffer is typically passed over the bound nucleic acid at least once, and generally two or more times. The number of times the bound nucleic acids are washed depends on numerous factors, such as the composition and volume of the lysate-alcohol mixture.

The glass-fiber filter containing the bound nucleic acids can optionally be dried. The glass-fiber filter can be dried by any method known to those skilled in the art. For example, the filter can be dried with heat at a temperature, typically less than 90° C., usually for one or more minutes. The drying condition is selected so as not to destroy or denature the nucleic acids. Other methods for drying the filter include, but are not limited to, air drying, use of a desiccator, etc.

After drying, the nucleic acids can be eluted from the filter by passing a suitable eluting solution through the filter. The eluting solutions preferably have low ionic strength. Thus, the concentration of salts and other ionic compounds in the eluting solution is kept to a minimum. An example of such an eluting solution is nuclease-free water, optionally containing about 0.01% to 2.00% Tween 20, optimally about 0.02% to about 0.1% Tween 20.

The nucleic acids can be eluted into a multiple-well collection plate placed below the multiple-well filtration plate (described above) and fitted to the vacuum or pressure manifold in such position that it can collect fluid samples that are passed through the filter. Multiple-well collection plates are commercially available. For example, a 96-well plate is sold by Becton Dickinson and Company (Franklin Lakes, N.J.) under the name Microtest.RTM. Alternatively, a tissue culture plate can be used.

The collection plate generally has wells that match those of the multiple well filtration plate and is fitted below the filtration plate in such a position as to collect the nucleic acids as they are passed through the glass-fiber filters. These plates, both the filtration plate and the collection plate, fit within the vacuum manifold in interlocking superposition.

Collection plates are readily commercially available. However, as with the 96-cell filtration plates discussed above, collection plates can also be adapted to have more or fewer wells of larger or smaller volumes depending on the needs of the user.

The extracted nucleic acids can be utilized in, for example, the methods described below.

The extraction method described above has been unexpectedly found to efficiently isolate both DNA and RNA. Other standard assays generally require one method for efficiently isolating DNA, and a separate method for efficiently isolating RNA.

Identifying a Pathogen in a Sample

In one embodiment, the invention provides a method for identifying a pathogen in a sample. The first step in the method is to extract nucleic acids from the cells or viruses in the sample. The nucleic acids are extracted in accordance with the extraction method as described above.

Once the nucleic acids are extracted from the sample, the next step in the method for identifying the pathogen is to assay the nucleic acids. The nucleic acids can be assayed by any method known to those skilled in the art.

The nucleic acids are typically subjected to nucleic acid amplification and/or to other standard analytical techniques. Nucleic acid amplification systems utilizing, for example, PCR or RT-PCR methodologies are known to those skilled in the art. For a general overview of nucleic acid amplification technology and a description of the application of these techniques for pathogen diagnosis see, for example, Dieffenbach et al., PCR Primer: A Laboratory Manual, Cold Spring Harbor Laboratory Press, New York (1995) and Clewley, The Polymerase Chain Reaction (PCR) for Human Viral Diagnosis, CRC Press, Boca Raton, Fla., Chapter 5 (1995).

Nucleic acid amplification systems that make use of PCR methodologies have already been automated. As discussed above, the method of the claimed invention for extraction of nucleic acids can also be automated. The automation of nucleic acid extraction and purification in conjunction with the automation of nucleic acid amplification technology enables the use of these methods to screen, in a short time, large numbers of samples, such as blood, for pathogens (e.g., viruses, bacteria, fungi or parasites, etc.) which can be present in the sample, even in extremely low levels.

The product of the nucleic acid amplification (amplicons) and thus, the identity of the pathogen from which the nucleic acids are derived, can be, for example, determined by hybridization techniques. Generally, hybridization techniques employ an oligonucleotide probe that is complementary to, and uniquely hybridizes with, a known nucleic acid sequence. The oligonucleotide probe may be an RNA or DNA molecule.

Any method for assaying hybridization of an oligonucleotide probe to a nucleic acid can be employed. For example, the technique of Southern hybridization (Southern blotting) is a particularly well known example of such a technique. The Southern blot technique involved cleaving the nucleic acid amplicons with restriction endonucleases, separating the cleaved fragments by gel electrophoreses, probing with a specific oligonucleotide probe, and detecting presence of hybridization. Other related methods are know to those in the art. See Sambrook et al., Molecular Cloning, A Laboratory Manual, 2d ed., Cold Spring Harbor Laboratory, Cold Spring Harbor (1989).

The length of the oligonucleotide probe is not critical, as long as it is capable of hybridizing to the target molecule. The oligonucleotide should contain at least six nucleotides, preferably at least ten nucleotides, and more preferably at least fifteen nucleotides. There is no upper limit to the length of the oligonucleotide probes. However, longer probes are more difficult to prepare and require longer hybridization times. Therefore, the probe should not be longer than necessary. Normally, the oligonucleotide probe will not contain more than fifty nucleotides, preferably not more than forty nucleotides, and more preferably not more than thirty nucleotides.

Such probes can be detectably labeled in accordance with methods known in the art, such as, for example, radiolabels, enzymes, chromophores, fluorophores, and the like. Detection of the label indicates hybridization of the oligonucleotide probe with the nucleic acid. Accordingly, detection of the label indicates that the sample contains the particular pathogen that the oligonucleotide probe is specific for, thus, identifying the pathogen in the sample.

Alternatively, failure to detect hybridization with a particular oligonucleotide probe for a specific pathogen indicates that the sample does not contain detectable amounts of nucleic acids for the particular pathogen that the oligonucleotide probe is specific for.

Another approach for assaying the nucleic acids is the use of conventional or universal molecular beacons. Such methods were described, for example, by Tyagi and Kramer (Nature Biotechnology 14, 303-308, 1996) and by Andrus and Nichols in U.S. Patent Application Publication No. 20040053284, which is assigned to the New York Blood Center, New York, N.Y.

Briefly, molecular beacons are single-stranded oligonucleotide hybridization probes that form a stem-and loop-structure. The loop contains a probe sequence that is complementary to a target sequence. The stem is formed by annealing complementary arm sequences on either side of the probe sequence. Typically, a fluorophore is covalently attached to the end of one arm and a quencher is covalently linked to the end of the other arm. The molecular beacons do not fluoresce when free in solution due to the stem-loop structure and the quenching of the fluorophore. However, when the molecular beacons hybridize to a nucleic acid containing a target sequence, the molecular beacon undergoes a conformational change that enables it to fluoresce.

The molecular beacon probes can be modified in any manner which allows for detection of the nucleic acids (e.g., PCR amplification products). Modified probes include, for example, the “wavelength-shifting” molecular beacon probes described in U.S. Pat. No. 6,037,130; and incorporated herein by reference. In particular, these modified probes have the basic molecular beacon probe structure, namely, a loop; stem duplex; a quencher on one end; and a reporter moiety, typically a fluorophore, opposite the quencher on the other end. The reporter is referred to as the “harvester reporter.” The modification of the probe is that the probe includes an extension of several nucleotides past the “harvester reporter.” The extension terminates in a nucleotide that is linked to an “emitter reporter,” typically another fluorophore. In the presence of the target nucleic acid molecule, the quencher separates from the reporters. In this open conformation the “harvester reporter” absorbs energy from the excitation source but transfers a significant portion of the energy, in some constructions the great majority of the energy, to the “emitter reporter,” which receives the transferred energy and emits it at its characteristic, longer wavelength.

Molecular beacons are typically sensitive to small numbers of nucleotide mismatches between a probe and a target sequence (Tyagi et al., 1998, Nat. Biotechnol., 16:49-53). The molecular beacon technology may be modified to permit their use in detection of, for example, even highly variant virus species. For example, U.S. patent application Ser. No. 10/399,843 by Andrus and Nichols (U.S. Patent Application Publication No. 20040053284, and assigned to the New York Blood Center) describes the use of forward and reverse primers which are aligned “nose-to-nose” on a target sequence (i.e., there is no intervening gap between the hybridization sites of the two primers). The molecular beacon probe is designed to hybridize asymmetrically across the junction of the two primers. PCR primers are capable of hybridizing to, and initiating amplification of target sequences in the presence of nucleotide mismatches. Due to the “nose-to-nose” configuration of the primers, the amplified PCR products share nucleotide sequence identity with the molecular beacon probe.

Thus, for instance, universal beacon RT-PCR assays can be developed to detect highly variant strains, including, for example, all major genotypes of HIV-1, such as Group O, and all subtypes of HCV. Examples of suitable universal beacon RT-PCR assays for detecting highly variant virus strains are described in U.S. patent application Ser. No. 10/399,843 (U.S. Patent Application Publication No. 20040053284).

In a preferred embodiment, multiplex PCR is employed. In this assay, the PCR mixtures contains primers and probes directed to the nucleic acids of multiple pathogens. Typically, a single fluorochrome is used in the assay. Thus, detection of a positive signal indicates that any one of the pathogens is present.

The identify of the specific pathogens detected can be determined by, for example, running individual PCR reactions directed to each of the pathogens being tested for, or by labeling the amplicon and hybridizing the amplicon to different immobilized targets. The targets can be immobilization to, for instance, nitrocellulose, DNA chips, microbeads, etc.

Other PCR-based methods can also be employed for assaying nucleic acids to identify pathogens in a sample. Examples of such methods include, gel analysis, Taqman®, etc.

Identifying Contaminants in an Aqueous, Non-Biological Sample

In another embodiment, the invention provides a method for identifying biological contaminants in an aqueous, non-biological (i.e., water) sample suspected of containing, or known to contain, cells, viruses, or both cells and viruses. Thus, water (e.g., drinking water, lakes, etc.) can be tested for biological contaminants to ensure the safety of the water for drinking or recreation (e.g., swimming). The biological contaminants can include cells or viruses. Examples of biological contaminants in water include bacteria (e.g., E. coli, fecal coliform, etc.), algae (e.g., blue-green algae, cyanobacteria, etc.), fungi (Phialophora sp., Exophiala sp.and Acremonium sp.), parasites (e.g., cryptosporidium, leishmania, Giardia lamblia, amoebae, flagellates, etc.), and viruses.

The first step in the method is to extract nucleic acids from the cells or viruses in the sample. The nucleic acids are extracted in accordance with the extraction method as described above.

To identify the contaminants, the nucleic acids can be assayed by, for example, PCR-based methods known to those skilled in the art. Suitable assay methods include those described above.

Identifying Genetic Disorders

In another embodiment, the invention provides a method for identifying genetic disorders in a mammal. The first step in the method is to extract nucleic acids from the cells in the sample. The nucleic acids are extracted in accordance with the extraction method as described above. Preferably, the cells are obtained from a mammal, usually a human.

To identify the genetic disorder, the nucleic acids are assayed by any method known to those skilled in the art. Suitable assay methods include those described above.

Any genetic disorder can be identified in accordance with the methods of the present invention. Examples of genetic disorders include, but are not limited to, hemophilia, sickle-cell anemia, down syndrome, cancer, predisposition to cancer (e.g, assaying for BRCA1 gene), tay-sachs, cystic fibrosis, cerebral palsy, Marfan syndrome, etc.

Kit

In another embodiment, the invention provides a kit for extracting nucleic acids from a sample. The kit comprises a lysing solution and glass-fiber filters. The lysing solution comprises a detergent. The kit optionally contains one or more of the following: alcohol, proteinase, washing buffer, elution buffer, and vessels (e.g., test tubes, multiple well plates, etc.).

The lysing solutions, detergents, glass-fiber filters, alcohol, proteinase, washing buffer, eluting buffer and vessels have been described above.

### EXAMPLES

### Example 1

### Materials

West Nile Virus (strain Hawaii) was obtained from the New York State Department of Health and cultured in Vero cells. The quantity of infective viral particles was determined by conventional plaque assay (Beaty et al, Arboviruses, p. 797-856. In N. J. Schmidt, and R. W. Emmons (ed.), Diagnostic procedures for viral, rickettsial and chalmydial infections. American Public Health Association, Washington, D.C. 1989). The amount of viral genome equivalents was ascertained by quantitative RT-PCR using a WNV RNA qualification panel, QWN702 (BBI Diagnostics, West Bridgewater, Mass.) as a standard for quantitiation.

The New York Blood Center provided HBV-infected plasma and plasma from a chronically HCV-infected blood donor. HIV stock was a cell-free supernatant from an HIV-infected human peripheral blood lymphocyte culture.

Normal human plasma, pre-tested for blood borne pathogens, was used for serial dilutions of positive or spiked samples.

### Example 2

### Extraction of Multiple Viral Genomes in Plasma

Aliquots of HBV-, HCV-, HIV- and WNV-samples were pipetted at various volumes into 96 well 2.2 ml storage plates (ABgene, Surrey, KT, UK). Plasma volumes used for direct extraction ranged from 150 to 450 μl.

Proteinase K (Qiagen, Chatsworth, Calif.) and AL lysis buffer (Qiagen, Chatsworth, Calif.) were added at optimized amounts (Table 1) and briefly mixed. The AL lysis buffer contains inter alia a detergent and guanidinium salts. After incubating the plates in a shaking water bath for 25 minutes at 58° C., predetermined amounts of absolute ethanol (Table 1) were gently mixed with the lysate. The above preparation was transferred to a 0.7 μm glass-fiber-filter plate (GF/F, Whatman, Clifton, N.J.) and filtered at −450 mm Hg vacuum. Depending on the total volume of the lysate preparation, the transfer was accomplished in one to three pipetting steps. The loaded filter plate was then washed with AW2 washing buffer (Qiagen, Chatsworth, Calif.) at the same vacuum setting. Washing volumes and repeats of washings depended on the initial sample volume (Table 1). After washing, a vacuum of −350 mm Hg was applied to the filter plate for 10 min to remove residual washing solution. Subsequently, the plate was kept at room temperature for 10 min with no vacuum to allow final air-drying. Purified nucleic acids were eluted into a U-bottom 96-well plate or PCR plates, by −350 mm Hg vacuum filtration of 65-100 μl nuclease free water containing 0.05% Tween 20.

TABLE 1
Conditions for extracting various plasma volumes with the GF/F method

| | Protocol | | |
|---|---|---|---|
| | I | II | III |
| Plasma | 150 μl | 300 μl | 450 μl |
| Proteinase K | 20 μl | 40 μl | 60 μl |
| AL lysis buffer | 200 μl | 400 μl | 600 μl |

Mix, incubation at 58° C. for 25 min

| | | | |
|---|---|---|---|
| Ethanol | 200 μl | 400 μl | 600 μl |
| Total lysate vol | 570 μl | 1140 μl | 1710 μl |

Mix, transfer to GF/F filter plate
Vacuum filtration at 450 mm Hg

| | | | |
|---|---|---|---|
| Washing volume | 600 μl | 1200 μl | 1600 μl |

Vacuum filtration at 450 mm Hg

| | | | |
|---|---|---|---|
| Washing steps | 1 | 3 | 3 |

Vacuum drying of filter plates at 350 mm Hg for 10 min
Air drying of filter plated plates with no vacuum for 10 min

| | | | |
|---|---|---|---|
| Elution | 65-100 μl | 65-100 μl | 65-100 μl |

1 min contact time before filtration at 350 mm Hg for 1 min

Pipetting and filtration was either manual or automated using Genesis RSP 150 and Genesis Workstation 200 (Tecan, Maennedorf, Switzerland).

Nucleic acid amplification and detection were achieved following in-house PCR reaction and cycle conditions as described by Lee et al (Stabilized viral nucleic acids in plasma as an alternative shipping method for NAT. Transfusion 42, 409-413, 2002).

Molecular beacon technology (Tyagi and Kramer, Molecular beacons: probes that fluoresce upon hybridization. Nature Biotechnology 14, 303-308, 1996) was employed to detect and quantify PCR amplicons. Primers and molecular beacons were designed suitable for detecting all common strains of HCV, HIV and WNV. The targets for the primers were located in the 5′-UTR untranslated region of HCV and West Nile virus (WNV), the gag- and pol-gene of HIV, and genes encoding surface antigen for HBV.

Light emission was monitored during every thermal cycle at the annealing step. The Sequence Detection v 1.6.3 software program (PE-Biosystems, Foster City, Calif.), determines the copy number of the target template by analyzing cycle-to-cycle change in fluorescence signal as a result of the amplification of template during PCR, and by comparing unknowns to a curve generated from serially diluted known synthetic RNA or plasmid DNA standard samples. All standards were calibrated with EUROHEP panels (CLB, Netherlands) for determination of copy numbers.

Release of both, viral RNA and DNA, from the protecting capsid and envelope was achieved by using AL lysis buffer in combination with proteinase K. Stability of RNA in AL lysis buffer was evaluated and found to be comparable with guanidine thiocyanate (data not shown). Nucleic acid capture, washing and elution were achieved by vacuum filtration through glass fiber membranes. Results are shown in Table 2.

TABLE 2
Comparison of plasma viral load, determined by using the GF/F protocol
versus Qiagen kits.

| | | GF/F² | | Qiagen³ | | |
|---|---|---|---|---|---|---|
| Virus | Dilution¹ | log₁₀ c/ml⁴ | SD | log₁₀ c/ml⁴ | SD | G/Q⁵ |
| HBV | 10⁻³ | 6.82 | 0.11 | 6.57 | 0.11 | 1.75 |
| | 10⁻⁴ | 5.94 | 0.04 | 5.51 | 0.11 | 2.64 |
| | 10⁻⁵ | 4.87 | 0.08 | 4.58 | 0.12 | 1.97 |
| HCV | 10⁻¹ | 5.47 | 0.12 | 5.20 | 0.17 | 1.88 |
| | 10⁻² | 4.49 | 0.16 | 4.20 | 0.17 | 1.94 |
| | 10⁻³ | 3.38 | 0.19 | 3.11 | 0.13 | 1.85 |
| HIV | 10⁻² | 7.55 | 0.09 | 7.56 | 0.06 | 0.98 |
| | 10⁻³ | 6.58 | 0.08 | 6.52 | 0.04 | 1.15 |
| | 10⁻⁴ | 5.56 | 0.09 | 5.77 | 0.18 | 0.62 |

¹Dilutions of positive samples in normal human plasma. The series of 10-fold dilutions for each virus was selected to cover a range of 10³ and 10⁸ copies/ml.
²150 μl plasma were extracted by proteinase K/AL buffer lysis and filtration through Whatman 96-well glass-fiber-filter plates (n = 8).
³140 μl HCV and HIV plasma were extracted using the QiaAmp RNA kit. 200 μl HBV plasma were applied for the QiaAmp DNA kit (n = 8).
⁴The genome quantity was determined by quantitative real time PCR and expressed in log₁₀ copies/ml.
⁵GF/F results were divided by the results obtained with Qiagen extraction kits to determine the relative extraction efficiency of the glass fiber method compared to the reference method.

### Example 3

### Reproducibility of Nucleic Acid Extraction

Repeatability of these quantitative test results were evaluated for intra-assay and inter-assay variation. Table 3 shows, for HCV as an example, coefficients of variation calculated for PCR results obtained after GF/F and Qiagen extraction. Intra-assay variations are similar for both procedures. However, the inter-assay PCR results were considerably less consistent for the Qiagen method. Nucleic acid recovery after manual, as well as automated extraction, proved to be consistent and reliable.

TABLE 3
Intra-assay and inter-assay variation determined for HCV PCR results
obtained after GF/F extraction and Qiagen extraction.

Coefficient of
Variation⁽¹⁾

| Extraction | Intra-assay | Inter-assay |
|---|---|---|
| GF/F method | 0.02 | 0.02 |
| Qiagen method | 0.02 | 0.05 |

⁽¹⁾Coefficient of variation was calculated for a set of 8 values each. Tests were performed by 4 different technicians at different times using aliquots of the same HCV infected plasma (diluted 1/100 in normal human plasma).

### Example 4

### Effect of PEG on Nucleic Acid Recovery

Aliquots of WNV-samples were pipetted into 96 well 2.2 ml storage plates (ABgene, Surrey, KT, UK) and mixed with various amounts of a 37% PEG 8000 stock solution. The total volume of the plasma-PEG preparation was 2.0 ml per extraction. After mixing PEG with the sample, a contact time of 10-30 min at RT was allowed before spinning at 1500 g for 3 minutes. The volume was then reduced to 200 μl by discarding 1.8 ml of the supernatant and saving the visible pellet including some remaining fluid (10× volume reduction).

40 μl proteinase K (Qiagen, Chatsworth, Calif.) and 270 μl AL lysis buffer (Qiagen, Chatsworth, Calif.) were added and mixed. Plates were then incubated in a water bath for heat digestion at 58° C. for 25 minutes. Thereafter, 270 μl absolute ethanol were gently mixed with the lysate. The extraction preparation of a total volume of 780 μl was transferred onto a 0.7 μm glass-fiber-filter plate (GF/F, Whatman, Clifton, N.J.) and filtered at −450 mm Hg. The loaded filter plate was washed once with 600 μl AW2 washing buffer (Qiagen, Chatsworth, Calif.) at the same vacuum setting. After washing, a vacuum of −350 mm Hg was applied to the filter plate for 10 min to remove residual washing solution. Subsequently, the plate was kept at room temperature for 10 min with no vacuum to allow final air-drying. Purified nucleic acids were eluted into U-bottom 96-well plates by −350 mm Hg vacuum filtration of 100 μl nuclease free water containing 0.05% Tween 20. Alternatively, elution was performed with 75 μl Tween-water filtrated directly into MicroAmp optical 96-well reaction plates (PE Applied Biosystems, Foster City, Calif.).

Pipetting and filtration were performed either manually or automated by means of a Genesis RSP 150 and Genesis Workstation 200 (Tecan, Research Triangle Park, N.C.).

In order to achieve maximum sensitivity, “high-volume” PCR reaction was carried out using 50 μl of the 100 μl eluate or, respectively, the total volume of the 75 μl eluate. For cDNA synthesis we added 30 μl reverse transcription (RT) mix (Table 4). The reaction was performed at 42° C. for 45 min followed by 95° C. for 2 min. Thereafter, 40 μl of PCR master-mix (Table 3) were added. Taq polymerase was activated at 95° C. for 10 min and target cDNA was amplified during 45 cycles of three thermal steps (95° C., 58° C. and 72° C.) of 30 seconds each. RT mix and PCR master-mix were optimized specifically for the 120 μl total reaction volume.

TABLE 4
RT mix and PCR mix for large volume amplification

| | | | |
|---|---|---|---|
| | | | μl/ |
| Reagent | Supplier | Concentration | sample |

30 μl RT mix per reaction

| | | | |
|---|---|---|---|
| M-MLV RTBuffer | Invitrogen | 5× | 16.0 |
| DTT | Gibco | 100 mM | 4.0 |
| PCR nucleotide mix | Amersham | 10 mM | 2.0 |
| | Biosciences | | |
| Reverse primer | | 100 μM | 1.0 |
| Rnase inhibitor | Promega | 4 U/μl | 0.4 |
| M-MLV Reverse | Invitrogen | 200 U/μl | 0.4 |
| transcriptase | | | |
| Nuclease-free water | Promega | 1× | 6.2 |

40 μl PCR mix per reaction

| | | | |
|---|---|---|---|
| MgCl2 | PE Applied | 25 mM | 3.0 |
| | Biosystems | | |
| Forward primer | | 100 μM | 0.1 |
| Molecular beacon | GeneLink | 200 ng/μl | 1.0 |
| TaqGold | PE Applied | 5 U/μl | 0.5 |
| | Biosystems | | |
| Nuclease-free water | Promega | 1× | 35.4 |

Molecular beacon technology (Tiyagi and Kramer, Molecular beacons: probes that fluoresce upon hybridization, Nature Biotechnology 1996, 14, 303-308) was employed to reveal PCR amplicons. The target for the primers was located in the 5′UTR region. Reverse primer (540 -gct ctt gcc ggg ccc tcc tg-3′), forward primer (5′-gca cga aga tct cga tgt cta aga aac-3′) and molecular beacon (5′-FAM cgcacg atc tcg atg tct aag aaa cc cgtgcg DABCYL-3′) were designed suitable to detect all common strains of WNV.

ABI Prism 7700 and 7900 Sequence Detection System instruments (PE Applied Biosystems, Foster City, Calif.) were used for amplification and detection. Amplification products were either determined by quantitative real time PCR or by qualitative post-PCR analysis. For real time PCR the Sequence Detection v1.6.3 software program (PE-Biosystems, Foster city, Calif.) determines the copy number of the target template by analyzing cycle-to-cycle change in fluorescence signal as a result of the amplification of template during PCR. The post-PCR analysis measures the relative light units emitted before and after amplification. The cut-off value for the qualitative post-run analysis was calculated from the average signal of negative controls plus 3 standard deviations.

Various amounts of PEG 8000 were added to the plasma to concentrate virus or viral components. For sedimentation of the PEG-plasma precipitate we chose conditions, which produce loosely formed pellets of constant size which could easily be re-suspended in lysis buffer. After pelleting the precipitate, we reduced the volume to 200 μl and processed the sample. AL lysis buffer/proteinase K, ethanol precipitation, filtration, washing and elution were adapted and optimized for extraction of nucleic acid from PEG-plasma sediment.

The elution was performed with 100 μl or 75 μl Tween-water, respectively, to maximize yield of purified RNA. 50 μl of extracted nucleic acids were utilized in the PCR reaction.

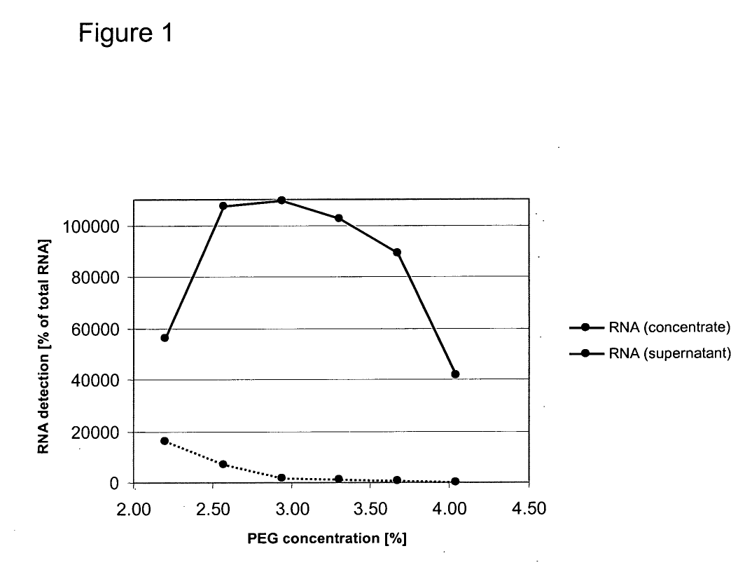

PEG 8000 was found to be an effective concentration method to enhance detection of WNV in human plasma samples. FIG. 1 demonstrates, that the highest amounts of viral RNA were determined in samples concentrated with 3% PEG 8000.

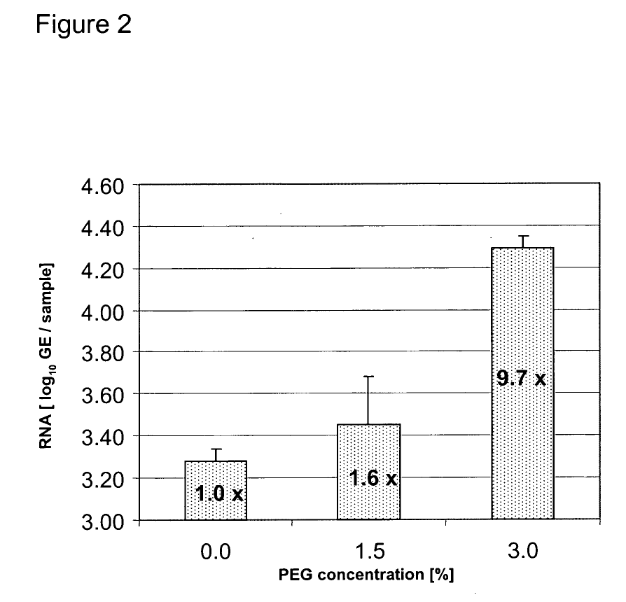

Using optimal conditions, a 10-fold increase of detectable RNA molecules was determined when concentrating and reducing the sample volume to 1/10 of the original PEG-plasma volume (FIG. 2). PEG produced similar concentration effects on HBV DNA when applied on HBV positive samples.

### Example 5

### Detection Limit

To evaluate the lower limit of detection, end-point titrations of plasma samples spiked with cultivated WNV were preformed. Quantity of RNA molecules and infective virions of the WNV preparation had previously been determined by quantitative PCR in comparison to the BBI panels and by plaque assay respectively. The quantity of viral genomes of our stock virus preparation was found to be 740 to 1500 times higher then the number of plaque forming viral particles.

To determine the sensitivity of the WNV assay, BBI stock (Uganda, 7.33×10⁴ copies/mL, Lot# 101702C) was diluted and tested. The BBI stock was diluted in negative plasma at 12.5, 6.3, 3.2, 1.6 and 0.8 copies per mL. Eighty replicates per dilution were tested and analyzed by Probit analysis to determine the 95% and 50% limit of detection (LOD).

Typical results are shown in Tables 5, 6, 7, and 8 below.

TABLE 5
FAM (WNV)

| | | | WNV Copies/mL | | | | | | | | | |
|---|---|---|---|---|---|---|---|---|---|---|---|---|
| | Controls | | 0.8 | | 1.6 | | 3.2 | | 6.3 | | 12.5 | |
| A | 952 | 925 | 1148 | 1035 | 1029 | 1300 | 1855 | 1675 | 1698 | 1602 | 2319 | 2209 |
| B | 794 | 893 | 1069 | 1082 | 1141 | 1038 | 2281 | 1807 | 1726 | 2069 | 2062 | 2496 |
| C | 821 | 806 | 939 | 864 | 1158 | 1284 | 1425 | 1808 | 2009 | 1536 | 2349 | 2178 |
| D | 799 | 944 | 1261 | 1101 | 1047 | 909 | 1192 | 1597 | 1347 | 1696 | 2295 | 2471 |
| E | 748 | 2900 | 1075 | 841 | 999 | 1221 | 1547 | 1361 | 1740 | 1845 | 2133 | 1669 |
| F | 674 | 1702 | 932 | 983 | 769 | 1106 | 1593 | 1443 | 1511 | 1364 | 1754 | 2001 |
| G | 728 | 698 | 922 | 825 | 900 | 1035 | 1418 | 1380 | 1567 | 1559 | 2191 | 1858 |
| H | 694 | 781 | 1103 | 1215 | 872 | 1074 | 1712 | 1641 | 1847 | 1421 | 1500 | 2105 |

Wells 1A-H: Internal Control (IC) Negative.
WNV Cutoff: Mean of wells 2A-2D (Negative plasma) + 5SD = 1197.
Well E2: WNV positive control at ˜300 copies/mL (estimation).
Well F2: WNV positive control at ˜60 copies/mL (estimation).
Positive WNV results are shown in bold.

TABLE 6
Summary of sensitivity data using 5 WNV dilutions

| | # Positive/# tested | | | | |
|---|---|---|---|---|---|
| WNV dilutions* | Run # | | | | |
| (WNV copies/mL) | 1 | 2 | 3 | 4 | 5 |
| 0.8 | 0/16 | 1/16 | 0/16 | 2/16 | 1/16 |
| 1.6 | 7/16 | 3/16 | 6/16 | 6/16 | 1/16 |
| 3.2 | 15/16 | 15/16 | 12/16 | 13/16 | 12/16 |
| 6.3 | 16/16 | 16/16 | 16/16 | 16/16 | 16/16 |
| 12.5 | 16/16 | 16/16 | 16/16 | 16/16 | 16/16 |

*Dilutions were made from BBI stock (Uganda, 7.33E+04 copies/mL, Lot# 101702C).

TABLE 7
Individual Probit Analysis (SPSS 11.5) and coefficients of variation for
these experiments is shown below:

| LOD % | Expt | Expt | Expt | | | | | |
|---|---|---|---|---|---|---|---|---|
| Detection | 1 | 2 | 3 | Expt 4 | Expt 5 | Mean | SD | CV |
| 95% | 3.09* | 3.36 | 4.07 | 4.06 | 4.18 | 3.75 | 0.49 | 0.13 |
| 50% | 1.92 | 2.13 | 2.33 | 2.08 | 2.64 | 2.22 | 0.28 | 0.12 |

*WNV copies/ml
LOD = Limit of detetion

TABLE 8
Overall Probit Analysis (SPSS 11.5) calculated from 80 replicates
of each dilution tested on 5 plates run on different days

| LOD | |
|---|---|
| % Detection | Copies/mL |
| 95% | 3.79 |
| 50% | 2.22 |

### Example 6

### Specific Description of the West Nile Virus Assay

Universal-Beacon RT-PCR for Detection of WNV. Primers and probes were targeted to a 47 nucleotide-long region spanning the junction between the 5′-untranslated region and the nucleocapsid start-site of the WNV genome. The reference sequence used for primer design and nucleotide numbering was the New York 1999-equine isolate reported by Lanciotti et al (Science 286:2333-2337, 1999; Genbank AF196835). The 47 nucleotide target region is >97% conserved within all Lineage 1 isolates for which Genbank sequence information is available, and is 94% identical to WNV Uganda 1937 (Genbank M12294). Primer and probe sequences are as follows: Forward Primer: 5′-GCACGAAGATCTCGATGTCTAAGAAAC-3′ (27mer, positions 83-109; 44% G/C; Tm 77° C.) and Reverse Primer: 5′-GCTCTTGCCGGGCCCTCCTG-3′ (20 mer, positions 110-129; 75% G/C; Tm 84° C.). Molecular Beacon Probe: 5′6-FAM-cgcacgATCTCGATGTCTAAGAAACCcgtgcg-DABCYL-3′ (WNV probe region is in upper case and stem nucleotides are in lower case).

RT-PCR for Dengue Virus Type 1 Internal Control RNA. Primers were designed to amplify a 67 b.p. region (nucleotides 10632-10698 of the 3′ non-coding region of Dengue Type 1 RNA (reference sequence Genbank AF513110). A VIC-labeled Taqman probe was used for control RNA PCR product detection to permit efficient discrimination between IC and WNV fluorescent signals in the ABI PRISM 7900HT. Primer and probe sequences are as follows: Forward Primer: 5′-GCATATTGACGCTGGGAGAGA-3′ (20 mer, positions 10632-10652; % G/C; Tm 73° C.) and Reverse Primer: 5′-GCGTTCTGTGCCT-3′ (13 mer, 10686-10698; 52% G/C; Tm 51° C.). Taqman probe 5′-VIC-AGATCCTGCTGTCTCTACA-MGB-3′ (19 mer, positions 10657-10675; 47% G/C; Tm 59° C.).

Sample source. Frozen plasma samples were tested. The samples are derived from whole blood collected in CPDA-1 anticoagulants from blood donors. They are centrifuged and then stored at −80° C. in 96-deep well plate. Stored plasma samples are thawed at 4° C. for 40-48 hours prior to use. This procedure has been found to retain all WNV RNA.

Sample preparation. Sample extraction is performed on two liquid handling systems (Tecan, Genesis RSP 150 and Genesis Workstation 200). 400 μL plasma samples are robotically transferred from an Archive plate to a new 96-deep well plate. Four WNV negative controls, one WNV positive control and three internal control (IC) negatives were robotically placed in the deep well plate. The internal control target RNA is mixed with lysis buffer prior to use. During the extraction it is processed throughout the entire procedure in the same manner as the WNV samples. Proteinase K and AL lysis buffer are mixed with the plasma samples on the Genesis RSP 150.

The mixture is incubated at 58° C. in a shaking water bath. After incubation, ETOH is added to the lysates on the Genesis Workstation 200. The mixture is then transferred to a 96-well glass fiber filter plate and vacuum filtrated for nucleic acids binding. The filter is washed twice successively by filtration. The filter is vacuum-dried and then air-dried. The WNV RNA is eluted by filtration with nuclease-free H₂O. The eluate is collected directly into the corresponding well of the PCR plate containing Reverse Transcription (RT) mix.

Amplification: Reverse transcription (RT) and PCR. The PCR plate created above which contains RT Master Mix and the eluted sample, is incubated for reverse transcription on an Applied Biosystems Model 2700 thermocycler. At the end of the RT step, reverse transcriptase is heat inactivated and PCR mix is added to each well. The PCR reaction mixture is first heated to activate AmpliTaq gold and PCR is then conducted for 45 cycles.

Detection: Fluorescence reading and calculations. A spectrofluorometric thermal cycler (ABI PRISM 7900HT, PE-Biosystems, Foster City, CA) is used for end-point detection at the end of 45 PCR cycles. WNV signal is detected with FAM labeled probe, which is read at 522 nm, and IC signal labeled with VIC which is read at 554 nm. Test runs are considered valid if both negative and positive control values fall within pre-determined ranges. The run is valid if positive and negative samples are in acceptable ranges. Results for individual samples are considered valid if the internal control (VIC) RFU value exceeds IC cutoff. The IC cutoff is calculated from the mean relative fluorescence unit (RFU) of IC negative controls plus 3 SD (n=3). The WNV reactive cutoff is calculated from the mean RFU of the WNV negative controls plus 5 SD (n=4). The samples with lower RFUs than IC cutoff will be considered as IC failures unless WNv PCR positive. A positive sample is one in which the RFU is greater than or equal to the WNV cutoff RFU regardless of IC RFU.

Positive Control Reagents. WNV Positive control WNV tissue culture supernatant was inactivated by heating at 60° C. for 1 hour, diluted in negative human plasma (1000 times), and quantitated by RT-PCR assay using a panel of samples containing known amounts of WNV RNA. The positive control sample is adjusted to contain 60 RNA copies/ml, rapidly frozen, and stored single use aliquots at −80° C. or below. Aliquots are thawed by shaking in a 37° C. water bath on the day of use.

Internal Control Reagents. Dengue (Hawaii strain) culture supernatant was inactivated by heating at 60° C. for 1 hour, diluted to 10⁷ pfu/mL in PBS, 10% negative human plasma and aliquoted in 80 uL amounts sufficient for use as internal control for a single or multiple plates. Aliquots are rapidly frozen and stored at −-80° C. or below, and thawed by shaking in 37° C. water bath on the day of use.

Procedure Summary. The assay is carried out with 400 uL of plasma by lysis of virions with an AL lysis buffer/proteinase K lysis solution. Dengue virus is used as an internal PCR control. The lysate is then absorbed under vacuum onto a glass fiber plate, which is washed and dried before eluting the nucleic acids for reverse transcription and PCR. All pipetting steps are performed on Tecan Genesis RFP 150 and 200 workstations. PCR amplification is performed on ABI Model 2700 thermocyclers. Amplified nucleic acids are detected in an ABI 7900 fluorescence reader. In addition the internal control, assay controls include negative controls and a WNV RNA positive control with a known number of copies of RNA. WNV RNA positivity is ascertained using an end point calculation in which the fluorescence in the test sample is compared to that of the negative controls.

Sample Source and Preparation. Plasma from CPDA-1 anticoagulated blood which were frozen at −80° C. is the source material for this particular study. Samples may be thawed at 4° C. for 40-48 hours and stored at 4° C. prior to use.

Positive, negative, and internal controls. For the screening assay, one positive control well containing heat inactivated WNV virus corresponding to 300 WNV RNA copies per milliliter will be used. Four wells contain WNV negative control plasma and three additional wells are set up with IC negative control plasma which will be processed with lysis buffer lacking the dengue internal control target RNA. Positive cut off for the WNV is calculated as Mean+5SD of the four WNV negative controls. Positive cut off for the dengue internal control is calculated as Mean+3SD of the three IC negative controls lacking dengue virus.

Lysis of virions. Virions contained in the plasma samples are lysed by the addition of Proteinase K and AL lysis buffer and the released nucleic acids are protected by the lysis buffer-during incubation in a water bath at 58° C. for 25 minutes. Dengue virus internal control is added to the lysis buffer just prior to use to serve as an internal control for all steps of the procedure.

Isolation of WNV RNA. Absolute ethanol is added to the lysate and the sample is transferred robotically to a glass fiber filter plate and filtered under vacuum. The filter is then washed with wash solution to remove proteins and potential inhibitors of PCR, dried, and eluted with nuclease free water directly into 96 well PCR reaction plates.

Reverse transcription. The entire nucleic acid eluate is subjected to reverse transcription and PCR amplification. Reverse transcription mix containing 5× first strand buffer, DTT, dNTPs, RNase inhibitor, WNV reverse (WNV R) primer, dengue reverse (DR) primer and M-MLV reverse transcriptase is combined with extraction eluate and incubated for 45 minutes at 42° C. followed by 2 minutes at 95° C.

PCR amplification. PCR mix containing WNV forward (WNV F) primer, a FAM labeled “Universal Beacon” WNV probe (WNV P), dengue forward (DF) primer and a VIC labeled dengue internal control probe (DP), MgCl₂, PCR buffer and Taq polymerase (AmpliTaq gold) is added to the RT wells. The PCR reaction mixture is first heated at 95° C. for 10 minutes to activate AmpliTaq gold, then 45 PCR cycles were carried out at 95° C., 58° C., and 72° C. on an ABI 2700 thermocycler.

Detection of PCR products. As target sequences are amplified, the PCR products bind the loop structure of the FAM labeled WNV beacon probes, preventing the stem hybridized therefore FAM (reporter) and DABCYL (quencher) are far apart; and fluorescence is obtained. The FAM is exited at 490 nm and read at 522 nm. As for Dengue internal control, the VIC labeled probe anneals downstream from one of the primer sites and the VIC molecule is cleaved by the 5′ nuclease activity of Taq DNA polymerase as the primer is extended. The VIC signal increases as the probe releases cleaved VIC reporter from the probe during the target amplification. The VIC is exited at 490 nm and read at 554 nm.

### Example 7

### Stability of WNV in Plasma Stored at 4° C.

Serial dilutions of WNV samples (1000, 50, 250, 125, 62.5 GE/ml) were stored at 4° C. for 0, 7 and 14 days before they were quickly frozen/thawed. Sample volume is 350 μl each extraction, five replicates for each sample. Four replicates for each sample were tested. RNA extraction was performed using Glass fiber filter plates on Tecan Robotics; endpoint RT-PCR for WNV RNA was performed.

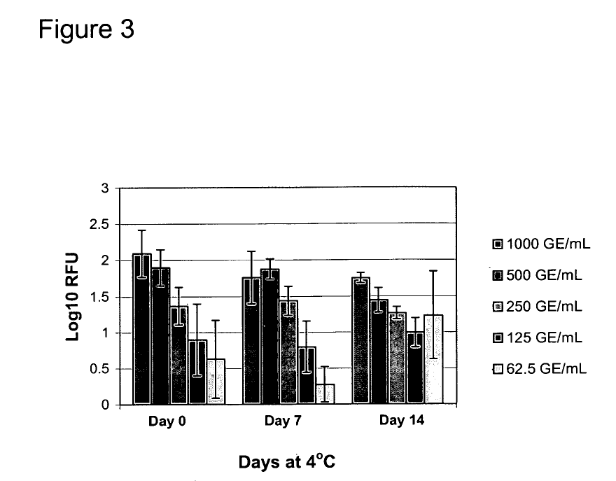

The results of RT-PCR are shown in FIG. 3, and a statistical analysis of the data is presented in Table 6. Samples stored at 4° C. for 7 to 14 days showed levels of WNV fluorescence signal which were equivalent to those of control samples stored at 4° C. for 0 days (Table 9). Thus, WNV RNA is stable in normal human plasma for at least 14 days when stored at 4° C.

TABLE 9
Statistical Analysis of Data Presented in FIG. 3

| | WNV detection | |
|---|---|---|
| Days at 4° C. | Mean* Log₁₀ RFU | t-test** |
| 0 | 1.92 ± 0.38 | |
| 7 | 1.76 ± 0.31 | P = 0.06 |
| 14 | 1.87 ± 0.36 | P = 0.34 |

*Data from all dilutions were back calculated to 1000 GE/mL (See FIG. 3).
**Data were analyzed by Student's T-test against Day-0 controls.

What is claimed is:

1. A method for extracting nucleic acids from a sample comprising:
a) obtaining a sample containing cells, viruses, or both cells and viruses;
b) adding a lysing solution comprising a detergent to the sample, thereby lysing the cells or viruses and forming a lysate;
c) adding an amount of alcohol to the lysate sufficient to aggregate or precipitate nucleic acids; and
d) purifying the nucleic acids from the lysate-alcohol mixture by filtering the mixture through a glass-fiber-filter.

2. A method according to claim 1, further comprising concentrating the cells or viruses in the sample.

3. A method according to claim 2, wherein the cells or viruses are concentrated with polyethylene glycol.

4. A method according to claim 1, wherein the sample is a biological sample.

5. A method according to claim 1, wherein the sample is an aqueous sample.

6. A method according to claim 1, wherein the sample contains a virus.

7. A method according to claim 1, wherein the cell is a microorganism.

8. A method according to claim 1, wherein the lysing solution further comprises a proteinase.

9. A method according to claim 8, wherein the proteinase is proteinase K.

10. A method according to claim 1, wherein the detergent is sodium dodecyl sulfate.

11. A method according to claim 1, wherein the detergent is tri-N-butylphosphate.

12. A method according to claim 1, wherein the sample is obtained from a mammal.

13. A method according to claim 12, wherein the mammal is a human.

14. A method according to claim 1, wherein the sample is obtained from a bird.

15. A method according to claim 1, wherein the sample is obtained from an arthropod.

16. A method for identifying a pathogen in a sample, the method comprising:
a) obtaining a sample containing cells, viruses, or both cells and viruses;
b) adding a lysing solution comprising a detergent to the sample, thereby lysing the cells or viruses and forming a lysate;
c) adding an amount of alcohol to the lysate sufficient to aggregate or precipitate nucleic acids;
d) purifying the nucleic acids from the lysate-alcohol mixture by filtering the mixture through a glass-fiber-filter; and
e) assaying the nucleic acids to identify the pathogen.

17. A method for identifying biological contaminants in a water sample, the method comprising:
a) obtaining a water sample containing cells, viruses, or both cells and viruses;
b) adding a lysing solution comprising a detergent to the sample, thereby lysing the cells or viruses and forming a lysate;
c) adding an amount of alcohol to the lysate sufficient to aggregate or precipitate nucleic acids;
d) purifying the nucleic acids from the lysate-alcohol mixture by filtering the mixture through a glass-fiber-filter; and
e) assaying the nucleic acids to identify the contaminants.

18. A method for identifying a genetic disorder in a mammal, the method comprising:
a) obtaining a biological sample containing cells;
b) adding a lysing solution comprising a detergent to the sample, thereby lysing the cells or viruses and forming a lysate;
c) adding an amount of alcohol to the lysate sufficient to aggregate or precipitate nucleic acids;
d) purifying the nucleic acids from the lysate-alcohol mixture by filtering the mixture through a glass-fiber-filter; and
e) assaying the nucleic acids to identify the genetic disorder.

19. A kit for extracting nucleic acids from a sample, the kit comprising:
a) a lysing solution comprising a detergent and
b) glass-fiber filters.
